(19) United States
(12) Patent Application Publication
(10) Pub. No.: US 2018/0070870 A1
(43) Pub. Date: Mar. 15, 2018

(54) Autonomous Sweat Extraction and Analysis Using a Fully-Integrated Wearable Platform

(71) Applicants: The Board of Trustees of the Leland Stanford Junior University, Palo Alto, CA (US); The Regents of the University of California, Oakland, CA (US)

(72) Inventors: Sam Emaminejad, Los Angeles, CA (US); Carlos Milla, Palo Alto, CA (US); Wei Gao, San Jose, CA (US); Ali Javey, Berkeley, CA (US); Eric Wu, Moraga, CA (US); Ronald W. Davis, Palo Alto, CA (US)

(21) Appl. No.: 15/700,119

(22) Filed: Sep. 9, 2017

Related U.S. Application Data
(60) Provisional application No. 62/385,405, filed on Sep. 9, 2016.

(51) Int. Cl. A61B 5/145; A61B 5/1477

(52) U.S. Cl. CPC A61B 5/14521; A61B 5/14532; A61B 5/1477

(57) ABSTRACT

A device for on-demand sweat extraction and analysis is realized as a printed circuit comprising a microcontroller, an iontophoresis circuit, a sensing circuit, and an electrode array having iontophoresis electrodes for sweat induction and sensing electrodes connected for sweat sensing. The sensing electrodes are positioned between the iontophoresis electrodes. The iontophoresis electrodes are preferably crescent-shaped and comprise a layer of agonist agent hydrogel loaded with sweat stimulating compounds. The iontophoresis circuit has a programmable current source for iontophoresis current delivery, and the sensing circuit includes two signal conditioning paths, where each of the paths includes an analog front-end to amplify a sensed signal and a low-pass filter to minimize high frequency noise and electromagnetic interference. The iontophoresis circuit and the sensing circuit are electrically decoupled for independent functionality.

### CROSS-REFERENCE TO RELATED APPLICATIONS

This application claims priority from U.S. Provisional Patent Application 62/385,405 filed Sep. 9, 2016, which is incorporated herein by reference.

### STATEMENT REGARDING FEDERALLY SPONSORED RESEARCH OR DEVELOPMENT

This invention was made with Government support under contract P01 HG000205 awarded by the National Institutes of Health. The Government has certain rights in the invention.

### FIELD OF THE INVENTION

The present invention relates generally to biosensors and biosensing techniques. More specifically, it relates to methods and devices for sweat extraction and analysis.

### BACKGROUND OF THE INVENTION

Wearable biosensors have received considerable attention owing to their great promise for a wide range of clinical and physiological applications. Despite significant progress made in printed and flexible biosensors in the field, a majority of wearable devices focus on monitoring of the physical activities or major electrophysiological parameters and only provide limited information regarding physiological changes of our complex biological systems. Wearable electrochemical sensors, which can measure the chemical compositions in body fluids, offer great opportunities for collecting physiological information at molecular levels. A fully integrated wearable sensing system for real-time monitoring of multiple analytes electrochemically in human perspiration during physical exercise has been developed which allows accurate measurement of sweat analytes through signal processing and calibration. However, for general population health monitoring and large-scale clinical investigations, on-demand sweat stimulation and in-situ analysis are required. Iontophoresis is currently a widely used method to stimulate local sweat secretion at a selected site and has shown great potential for a variety of clinical and physiological applications. For example, the sweat chloride level in iontophoresis extracted sweat sample is currently considered the gold standard for diagnosing cystic fibrosis, a chronic disease that affects lungs and digestive system. A strong correlation between blood and sweat ethanol concentrations has been reported which could enable continuous blood-alcohol monitoring by sweat analysis. Recent study also showed that the iontophoresis based extracted sweat contains glucose which can accurately reflect blood glucose. However, despite these advances, it remains a challenge to enable sufficient sweat extraction to provide accurate results.

### BRIEF SUMMARY OF THE INVENTION

Sweat analysis has been widely under-used mainly due to the fundamental physical barrier that exists in accessing this physiologically rich source of information. The present invention overcomes this barrier and allows for continuous and periodic sweat extraction and analysis on-demand.

Embodiments of the invention provide an autonomous wearable sweat extraction and analysis platform that periodically induces sweat with the aid of the iontophoresis process, and simultaneously and selectively measures a panel of target analytes in the extracted sweat. The approach overcomes one of the fundamental barriers in adoption of sweat-based sensing by making this physiologically rich source of information accessible on-demand. Hence, it enables a broad range of non-invasive diagnostic and general population health monitoring applications. The utility of the platform is demonstrated as both a diagnostic and investigative tool in the context of diagnosing cystic fibrosis and understanding the metabolic correlation of glucose content in sweat vs. blood.

Embodiments of the invention provide a system that implements a wirelessly programmable iontophoresis capability to induce sweat with different excretion rate profiles and at periodic time intervals. Through integration of sensing electrodes on the same substrate as that of the iontophoresis electrodes, the induced sweat can be analyzed on-site immediately. The sensors are capable of quantifying a panel of analytes in sweat with high sensitivity in the physiologically relevant range of interest.

In one aspect, the invention provides a device and method for sweat extraction and analysis in a miniature, wireless, programmable, wearable system. Embodiments include a novel electrode design for interfacing with the skin and a novel hydrogel design, which allow extraction of sweat in sufficient quantities to make such a miniature device possible. Embodiments also include periodically and programmably inducing the production of sweat for collection and automated analysis throughout the day. A technique for optimal stimulation of sweat glands allows production of sweat more than an order of magnitude larger than previously existing techniques. A design of stimulation and sensing electrodes reduces the chance of sweat evaporation between production and sensing.

In one aspect, the invention provides a device for on-demand sweat extraction and analysis. The device includes a printed circuit comprising a microcontroller, an iontophoresis circuit connected to the microcontroller, a sensing circuit connected to the microcontroller, and an electrode array. The electrode array comprises iontophoresis electrodes connected to the iontophoresis circuit for sweat induction, and sensing electrodes connected to the sensing circuit for sweat sensing, wherein the sensing electrodes are positioned between the iontophoresis electrodes. The iontophoresis electrodes comprise a layer of agonist agent hydrogel loaded with sweat stimulating compounds. The iontophoresis circuit comprises a programmable current source for iontophoresis current delivery. The sensing circuit includes two signal conditioning paths, where each of the paths includes an analog front-end to amplify a sensed signal and a low-pass filter to minimize high frequency noise and electromagnetic interference. The iontophoresis circuit and the sensing circuit are electrically decoupled for independent functionality. Preferably, the iontophoresis circuit comprises a current protection control circuit. The sweat stimulating compounds preferably comprise a cholinergic sweat stimulating compound. The iontophoresis electrodes preferably have crescent shapes having convex sides facing each other. Preferably, the iontophoresis electrodes comprise corrosion-proof contacts.

In some embodiments, the stimulation component can equivalently be used to perform “reverse iontophoresis” (different from iontophoresis) which enables extraction of interstitial fluid for in-situ analysis. The “reverse iontophoresis” operation can be achieved by using agonist-free hydrogels at the interface of current delivering electrodes and skin. By applying electrical current through the skin, we induce migration of charged ions to produce a convective solvent flow that transports uncharged species such as glucose towards the cathode. In some embodiments, the integration platform can equivalently be used to induce sweat thermally (by attaching a resistive element to the iontophoresis circuit).

In some embodiments, other agonist agents can be used to stimulate sweat production. Depending on the choice of the agonist, different patterns of sweat secretion can be achieved. In this work, we demonstrated various patterns of sweat secretion using three different cholinergic agonist hydrogels (acetylcholine, methacholine and pilocarpine) each at two different concentrations. The integrated system can also be used to quantify other target analytes in sweat, such as metabolites, electrolytes, heavy metals, and proteins.

In summary, embodiments of the invention provide a device and method for programmable, wireless sweat extraction on-demand using a wearable platform. It enables the induction of sweat at different rates with various patterns through the use of an integrated system. It allows periodic sweat induction using the same system/setup to enable periodic sampling and continuous monitoring. It also performs simultaneous sweat extraction and multiplexed analysis (seamless, eliminating contamination and evaporation issues).

The invention provides a fully integrated and autonomous platform that can stimulate sweat secretion and analyze the sweat content in-situ. The approach overcomes one of the fundamental barriers in adoption of sweat-based sensing for general population health monitoring by making this physiologically rich source of information accessible on-demand. As a result, it enables unprecedented applications in personalized medicine such as in-home continuous patient monitoring in response to potentially novel CF modulating drugs, and it spurs further clinical investigations including diabetes and pre-diabetes monitoring.

### BRIEF DESCRIPTION OF THE SEVERAL VIEWS OF THE DRAWINGS

FIG. 1A is a perspective view of an autonomous sweat extraction and sensing device configured to be worn on an arm, according to an embodiment of the invention.

FIG. 1B is a system-level block diagram of an autonomous sweat extraction and sensing device showing the iontophoresis and sensing circuits, according to an embodiment of the invention.

FIG. 1C is a cross-sectional illustration of the iontophoresis mode of operation of an autonomous sweat extraction and sensing device, according to an embodiment of the invention.

FIG. 1D is a cross-sectional illustration of a sensing mode of operation of an autonomous sweat extraction and sensing device, according to an embodiment of the invention.

FIGS. 2A-F are graphs showing experimental characterizations of an iontophoresis and sensing system, according to an embodiment of the invention.

FIG. 3A is a bar graph illustrating induced sweat secretion rate characteristics in response to three different custom-developed cholinergic agonist hydrogels with two different concentrations, according to an embodiment of the invention.

FIG. 3B are sweat rate profiles for periodic sweat induction comparing two different hydrogel concentrations and current durations, according to an embodiment of the invention.

FIGS. 4A-B are graphs of the real time on body continuous measurement of sweat sodium and chloride levels of a normal subject and CF patient, respectively, after iontophoresis based sweat stimulation.

FIG. 4C is a bar graph showing the comparison of sweat electrolyte levels between a group of normal subjects and a group of CF patients.

FIGS. 5A-G are bar graphs comparing blood and sweat glucose levels during fasting and 1 hour after glucose intake.

FIG. 6 is an illustration of flexible and wearable iontophoresis electrodes and electrochemical Na⁺ and Cl⁻ sensors, according to an embodiment of the invention.

FIG. 7A and FIG. 7B are schematic circuit diagrams showing analog sensor signal-conditioning circuitry, according to an embodiment of the invention.

FIG. 8 is a schematic circuit diagram showing the current delivery circuitry, according to an embodiment of the invention.

FIG. 9 is a graph of long-term continuous measurement of a Cl⁻ sensor in solutions containing 20, 40 and 80 mM NaCl, respectively.

FIG. 10 is a graph showing results of a repeatability study of the Ag/AgCl based Cl⁻ sensors in solutions containing 10, 20, 40 and 80 mM NaCl solutions, respectively.

### DETAILED DESCRIPTION OF THE INVENTION

We present an autonomous wearable sweat extraction and analysis platform that periodically induces sweat with the aid of the iontophoresis process, and simultaneously and selectively measures a panel of target analytes in the extracted sweat. Our solution overcomes one of the fundamental barriers in adoption of sweat-based sensing by making this physiologically rich source of information accessible on-demand. Hence, it enables a broad range of non-invasive diagnostic and general population health monitoring applications. Here, we demonstrated the potential utility of the platform as both a diagnostic and an investigative tool in the context of diagnosing Cystic Fibrosis and understanding the metabolic correlation of glucose content in sweat vs. blood.

Perspiration-based wearable biosensors facilitate continuous monitoring of individuals' health states and can collect physiologically-relevant information at molecular levels in real-time. Yet, the inaccessibility of human sweat has posed a fundamental bottleneck in adoption of sweat-based sensing as a non-invasive method of diagnosis and screening. For general population health monitoring and large-scale clinical investigations, on-demand sweat extraction and in-situ analysis is a necessity. Here, an autonomous sweat extraction and analysis wearable platform is presented that periodically induces sweat secretion with the aid of the iontophoresis process, and simultaneously and selectively measures a panel of target analytes in the extracted sweat. This platform includes a plastic based unit, containing the sweat induction and sensing electrodes, integrated into a wireless flexible printed circuit board. The circuit board consolidates the required IC chips and peripheral electronics to implement iontophoresis, signal processing and wireless transmission circuitries, thus, delivering a fully integrated system. Through performing on-body human subject testing, we demonstrated the utility of the platform as a diagnostic and clinical investigation tool. In particular, the system was used to induce sweat and detect the elevated sweat electrolyte content of Cystic Fibrosis patients as compared to that of healthy control subjects. Furthermore, we used the platform as an investigation tool to conduct preliminary studies toward understanding the metabolic correlation of glucose content in sweat vs. blood. Our results indicate that oral glucose consumption in fasting subjects results in increased glucose levels in both sweat and blood. Our solution enables a broad range of non-invasive diagnostic and general population health monitoring applications.

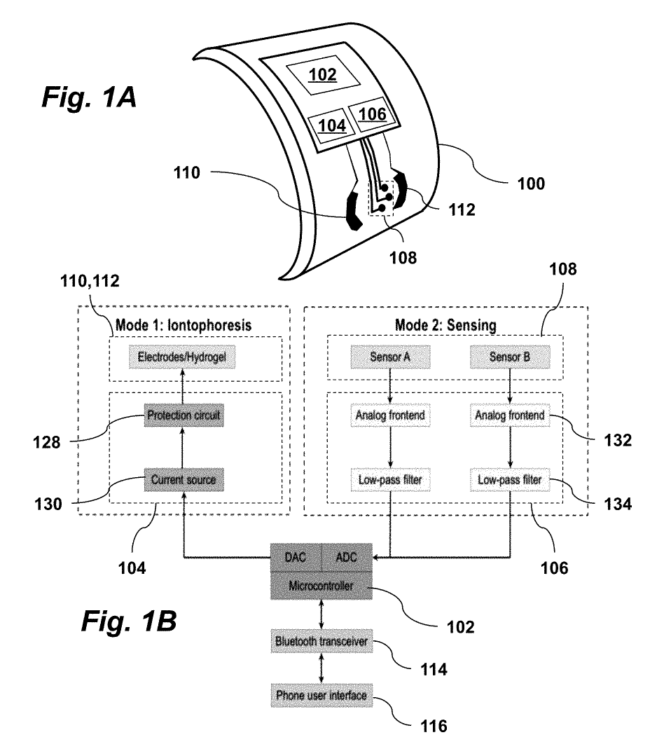

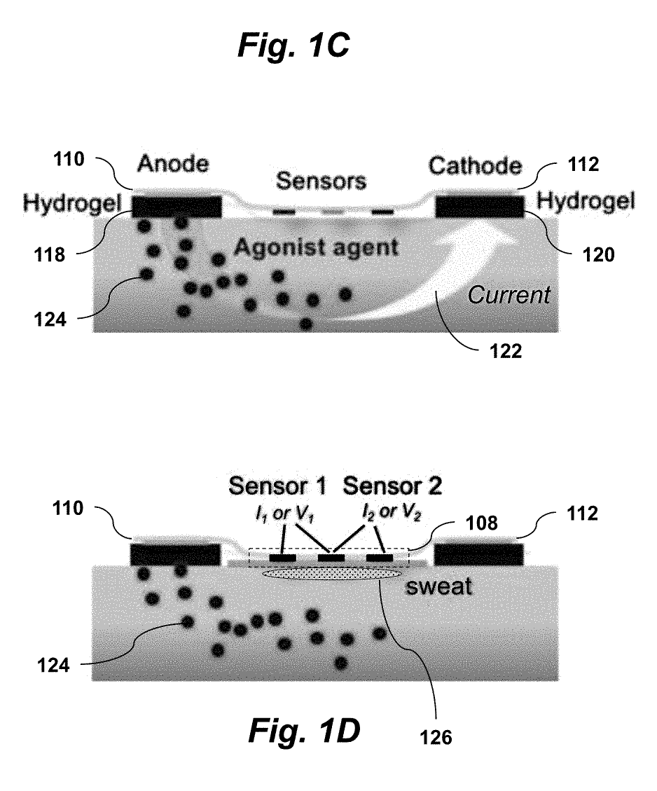

An embodiment of the invention, shown in FIG. 1A and FIG. 1B, provides a fully integrated and autonomous platform that can stimulate sweat secretion and analyze the sweat content in-situ. The device overcomes one of the fundamental barriers in adoption of sweat-based sensing for general population health monitoring by making this physiologically rich source of information accessible on-demand. As a result, it enables unprecedented applications in personalized medicine such as in-home continuous patient monitoring in response to potentially novel CF modulating drugs and fuels further clinical investigations including diabetes and pre-diabetes monitoring.

This system implements a wirelessly programmable iontophoresis capability to induce sweat with different excretion rate profiles and at periodic time intervals. Through integration of sensing electrodes on the same substrate as that of the iontophoresis electrodes the induced sweat can be analyzed on-site immediately. The sensors are capable of quantifying a panel of analytes in sweat, with high sensitivity in the physiologically relevant range of interest.

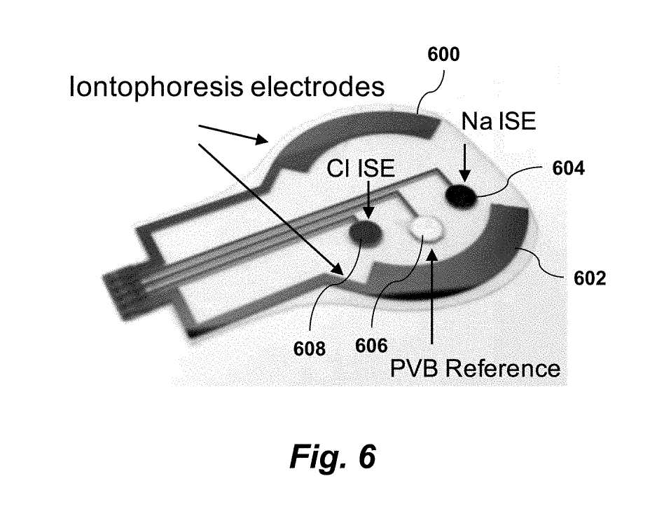

As shown in FIG. 1A and FIG. 1B, the device has an electrode array, containing the sweat induction electrodes 110, 112 and sensing electrodes 108, integrated into a wireless flexible printed circuit board (FPCB) 100. The independent functionality of the individual sensors and the iontophoresis process is preserved through electrically decoupling the switchable sweat sensing and sweat induction modes of operation. The electrodes are patterned on a plastic-based and mechanically flexible polyethylene terephthalate (PET) substrate to form a stable sensor-skin contact. Also integrated into the FPCB 100 are microcontroller 102, iontophoresis circuit 104, and sensing circuit 106. FIG. 6 shows more detail of the flexible and wearable iontophoresis electrodes and electrochemical Na⁺ and Cl⁻ sensors. Iontophoresis electrodes 600 and 602 each have a crescent shape, such that their concave sides face toward each other. For example, they may be shaped as opposite sectors of a common circle. More generally, the electrodes 600 and 602 may have any other shape or configuration that focuses the agonist delivery toward a region between them. The sensing electrodes 604, 606, 608 are positioned in between the two iontophoresis electrodes 600, 602.

Returning to FIG. 1A and FIG. 1B, sweat induction electrodes 110, 112 interface the skin with a thin layer of agonist agent hydrogel in between. To electrically connect the sweat induction electrodes and the hydrogels, thin stainless steel (corrosion proof) contacts are used. The hydrogels are loaded with cholinergic sweat stimulating compounds (e.g. pilocarpine). Depending on the devised compound formulation, different patterns of sweat rate can be achieved. The sensing electrodes interface the skin through a water-absorbent thin rayon pad. To demonstrate the sweat analysis capability, we developed potentiometric sodium and chloride sensors, functionalized with ion-selective films, as well as amperometric glucose sensor with the aid of glucose oxidase. The panel of target analytes was selected based on their informative role in terms of clinical diagnosis or providing understanding of an individual's physiological state. Specifically, sodium and chloride levels in sweat are diagnostic markers for Cystic Fibrosis and glucose level in sweat is reported to be metabolically related to that in blood.

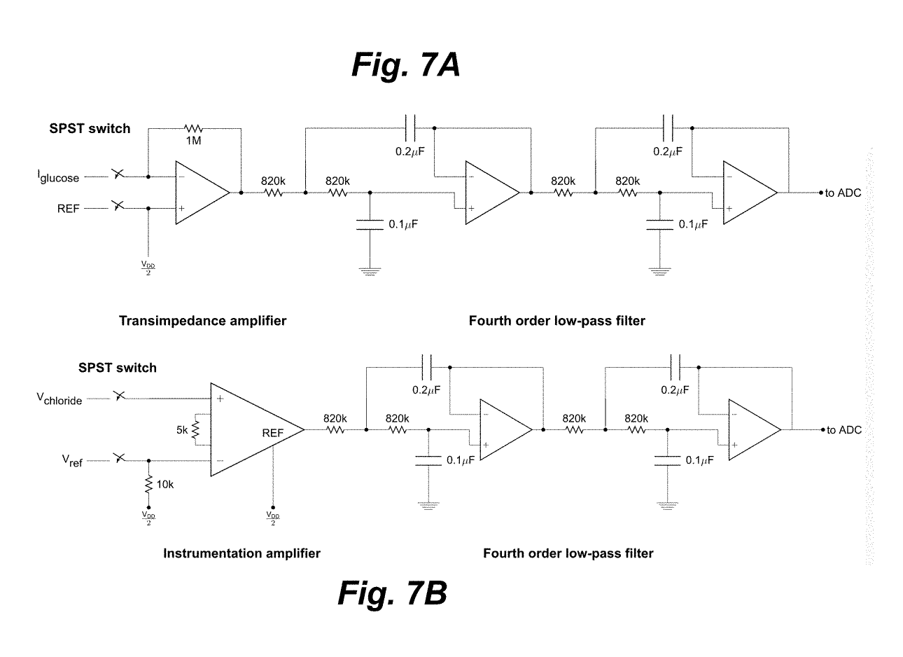

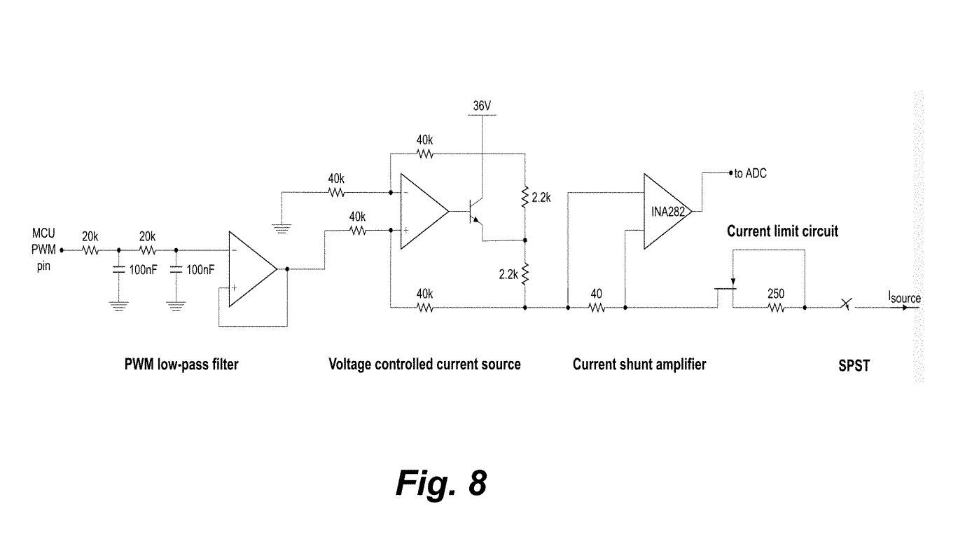

The circuits 102, 104, 106 are realized using IC chips and peripheral electronics to implement iontophoresis, signal processing, control and wireless transmission, thus, delivering a fully integrated, seamless and programmable system (see also FIG. 7A, 7B, FIG. 8).

FIG. 1B illustrates the system-level overview of the device, organized to illustrate induction and sensing modes of operation. The sweat induction circuit 104 includes a programmable current source 130 for iontophoresis current delivery and a protection circuit 128 that sets an upper limit on the iontophoresis current as a safety mechanism to avoid overheating and burning the skin. The sweat sensing circuit includes two signal conditioning paths in relation to the corresponding transduced signal, where each includes an analog front-end 132 to amplify the signal as well as a low-pass filter 134 to minimize the high frequency noise and electromagnetic interference (also see FIG. 7A FIG. 7B). The FPCB 100 includes a microcontroller 102 that can be programmed to set the mode of operation through controlling a bank of switches to turn on/off the respective circuits and electrical paths. The microcontroller's digital-to-analog (DAC) port is used to drive the iontophoresis circuit 104 and its analog-to-digital (ADC) port is used to convert the analog-processed signal from the sensing circuit 106 into the digital domain. The microcontroller 102 interfaces with an on-board wireless transceiver 114 to communicate the incoming/outgoing data from/to a Bluetooth-enabled mobile handset 116 with a custom-developed application. The mobile application has a user-friendly interface for programming the mode of operation as well as displaying and sharing the iontophoresis and sweat analysis data through email, SMS, and cloud servers.

FIG. 1C is a cross-sectional illustration of the iontophoresis mode of operation of an autonomous sweat extraction and sensing device. Anode 110 interfaces the skin with hydrogel 118, and cathode 112 interfaces the skin with hydrogel 120. A voltage between anode 110 and cathode 112 produces a current 122 in the skin and releases an agonist agent 124 into the skin. FIG. 1D is a cross-sectional illustration of a sensing mode of operation of an autonomous sweat extraction and sensing device. The agonist agent 124 and current 122 generated in the iontophoresis mode result in on-demand production of sweat 126 localized at the sensors 108 positioned between the electrodes 110 and 112.

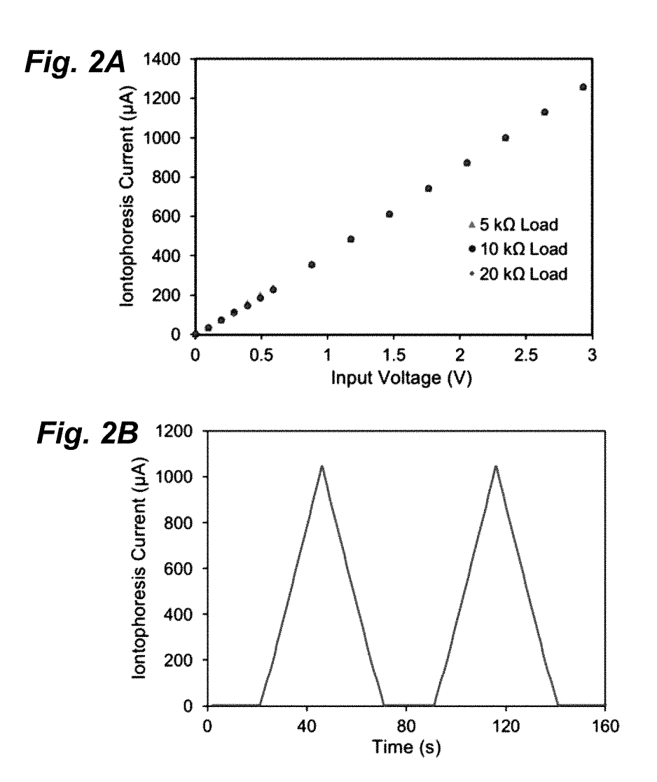

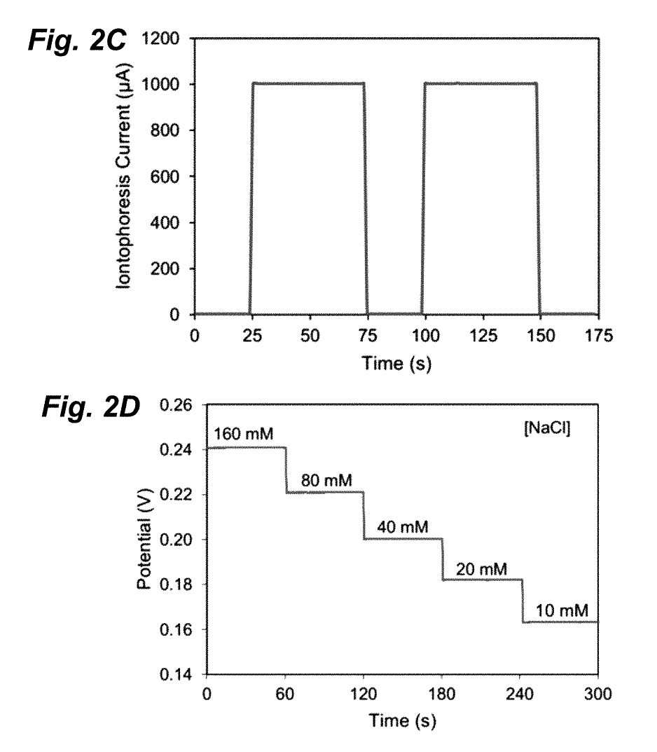

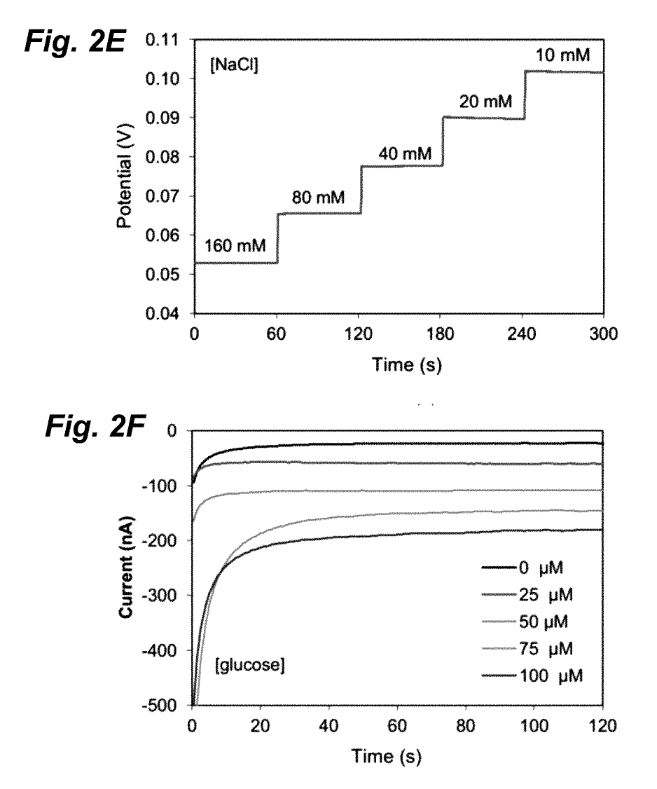

FIGS. 2A-F illustrate experimental characterizations of the iontophoresis and sensing system. FIG. 2A shows controlled iontophoresis current output for various resistive loads. FIG. 2B and FIG. 2C show programmed iontophoresis current to generate saw tooth and square wave patterns, respectively. FIG. 2D and FIG. 2E The open circuit potential responses of the sodium and chloride sensors, respectively, in NaCl solutions. FIG. 2F The chronoamperometric responses of a glucose sensor to glucose solutions.

The iontophoresis circuit 104 (FIG. 1A) was implemented as a digitally-programmable current source, ensuring that variation in the skin condition of individuals does not affect iontophoresis performance. FIG. 2A demonstrates the programmability and current source behavior of the circuit. The circuit delivers a current proportional to the output voltage of the microcontroller's digital-to-analog port, and this current is independent of load sizes ranging from 5 kΩ to 20 kΩ (the typical skin impedance in our context is ˜10 kΩ). The programmability of the current source circuit allows for inducing different iontophoresis current profiles, which in turn allows for sweat stimulation with controlled intensity and duration of sweat rate. FIG. 2B and FIG. 2C illustrate our platform's capability to generate iontophoretic currents with a sawtooth wave profile (FIG. 2B) and a square wave profile (FIG. 2C).

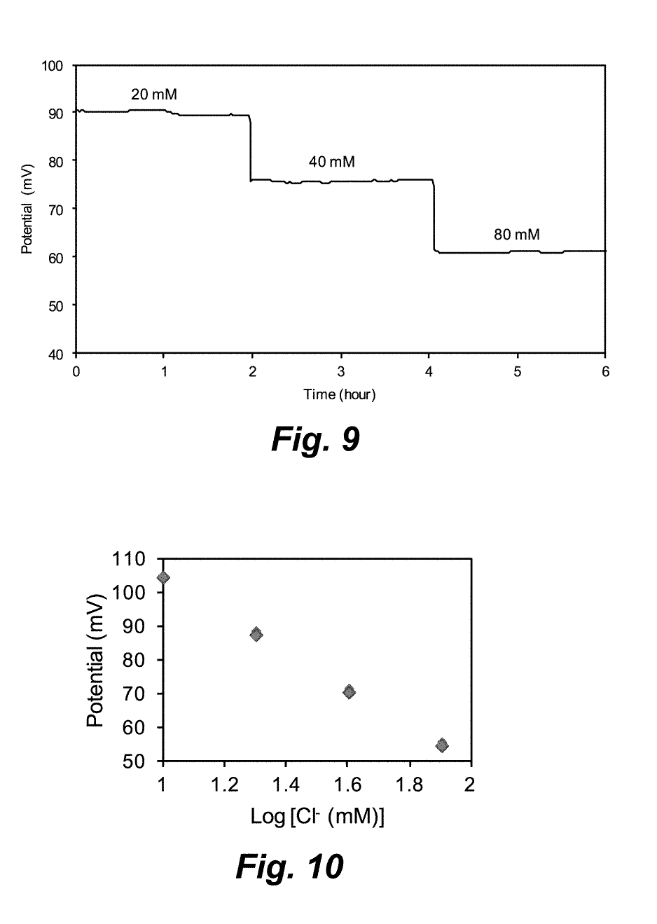

The sensing electrodes 106 (FIG. 1A) of the device can be modified differently according to the specific applications. FIG. 2D, FIG. 2E, FIG. 2F illustrate examples of the modified electrochemical sensors for sweat chloride, sodium and glucose analysis. Ag/AgCl electrodes were chosen for chloride ion detection due to their high selectivity while the measurement of sodium ions was achieved by using sodium ionophore X selectophore based ion selective electrode. A polyvinyl butyral (PVB)-coated electrode containing saturated chloride ions was chosen as the reference electrode due to its stable potentials in different analyte solutions. The performance of Na⁺ and Cl⁻ sensor was characterized in different NaCl solutions with physiological relevant concentrations. The potential differences between the ion selective electrodes and the PVB coated reference electrode were measured through a differential amplifier. FIG. 2D and FIG. 2E shows the representative voltage responses of the Na+ and Cl⁻ sensors, measured in 10-160 mM NaCl solutions, respectively. Both ion selective sensors show a near-Nerstian behavior with sensitivities of 63.2 mV and 55.1 mV per decade of concentration for Na⁺ and Cl⁻ sensors, respectively. FIG. 9 illustrates the long term continuous measurement a Cl⁻ sensor over a 6-hour period in 20, 40 and 80 mM NaCl solutions. The repeatability of the chloride sensors is demonstrated in FIG. 10. Three typical Cl⁻ sensors show nearly identical absolute potentials in 10-80 mM NaCl solutions with a variation of <1% in sensitivity. FIG. 2F shows the chronoamperometric responses of a glucose sensor to glucose solutions with typical sweat concentration range from 0 μM to 100 μM. The sensitivity of the glucose sensor is estimated as 2.1 nA/μM. Results of long-term stability studies of these electrochemical sensors indicate that the sensitivities of the biosensors are consistent over 2 weeks with sensitivity variations of <5%.

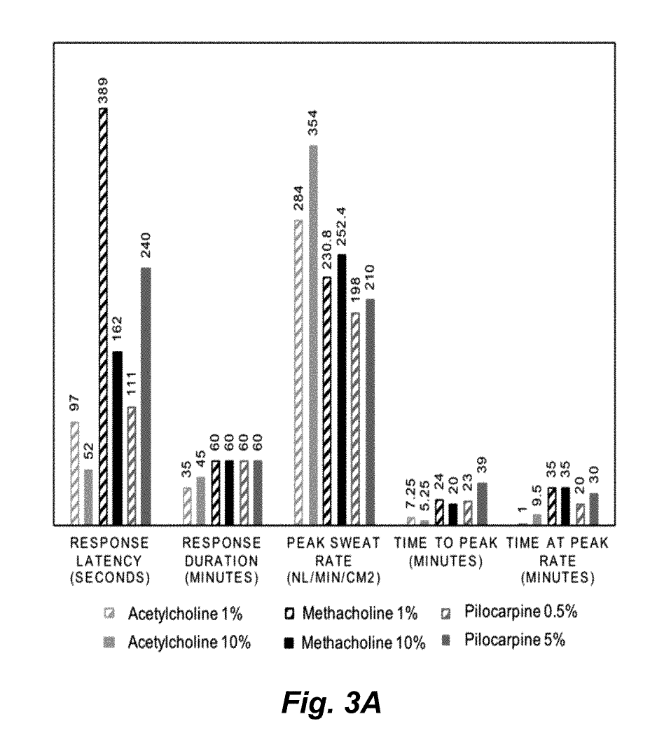

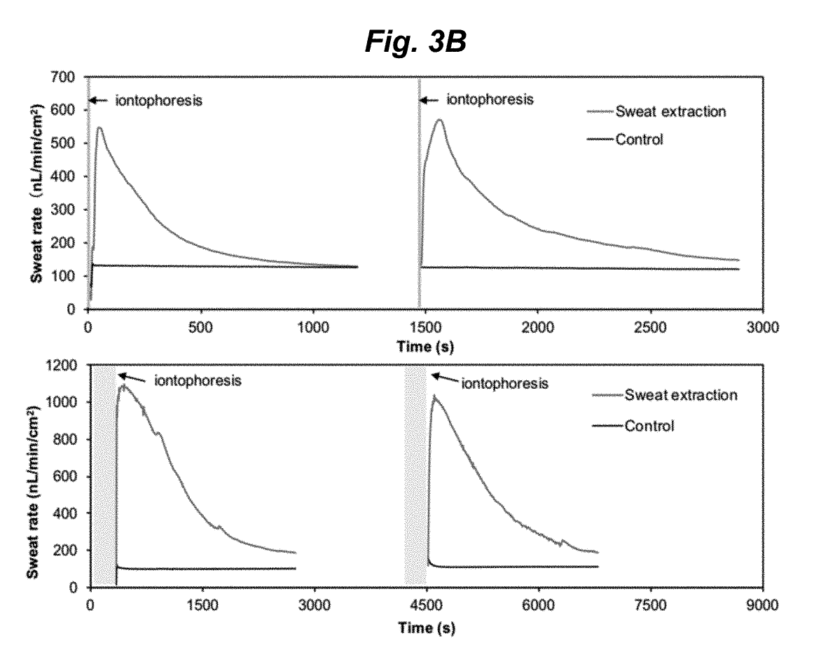

FIG. 3A shows induced sweat secretion rate characteristics in response to 3 different custom-developed cholinergic agonist hydrogels with 2 different concentrations: Acetylcholine, Methacholine, and Pilocarpine. Bars represent values for response latency (time in seconds to onset of secretion from start of iontophoresis), response duration (total time in minutes of secretion above baseline, measurements stopped at 60 minutes), peak secretory rate in response to stimulation, time to reach peak secretory rate and time spent secreting at the peak rate. FIG. 3B shows sweat rate profiles pertaining to periodic sweat induction using acetylcholine 1%-based hydrogel with iontophoresis current of 1 mA for 10 s (top panel) and acetylcholine 10%-based hydrogel with iontophoresis current of 1 mA for 5 min (bottom panel).

By modulating the formulation of the compounds that are loaded into the iontophoresis hydrogel, we can achieve different patterns of sweat secretion rate. We characterized the induced sweat rate profiles as stimulated by three different cholinergic agonist hydrogels (acetylcholine, methacholine and pilocarpine) each at two different concentrations. For this characterization step, 2 mA of current over duration of 5 minutes was applied using a pair of ring-shaped electrodes (WR Medical Electronics Co., MN, area: 4.3 cm2), with the sweat rate sensor (Q-sweat, WR Medical Electronics Co., MN) mounted on the positive electrode, sealing the stimulated area. As illustrated in FIG. 3A, for all of the formulations sweat secretion initiated in just a few minutes from the start of iontophoresis. In particular, acetylcholine-based presented a high sweat rate response with a short lifetime. This pattern is suitable for the case where periodic sweat sampling with short intervals is required. To demonstrate the periodic sweat stimulation capability we used our integrated platform and custom-developed acetylcholine-based hydrogel to induce sweat repeatedly in the same area. To retrieve the induced sweat rate information, immediately after each stimulation step, the stimulated area was wiped dry and sealed with the sweat rate sensor. After each characterization step, the same pair of hydrogels were reused for the subsequent stimulation. By modulating the duration of the applied iontophoresis as well as the concentration of the agonist agent we were able to tune the active sweat secretion window from a few minutes (FIG. 3B, top panel, acetylcholine 1%, iontophoresis current: 1 mA for 10 s) to 10s of minutes (FIG. 3B, bottom panel, acetylcholine 10%, iontophoresis current: 1 mA for 5 min).

Furthermore, our characterization results indicated that pilocarpine and methacholine-based hydrogels provide long duration of secretion beyond the 60-min characterization window, where about half of the secretion period were spent at about the peak rate. Specifically, methacholine at 10% concentration gave the optimal combination of a rapid onset of secretion with high secretory rate and sustained secretion at high rate that is also above the minimum recommended for sweat chloride analysis in CF (>100 nL/cm²/min). Therefore, for subsequent on-body sweat extraction and sensing experiments we used this formulation for our hydrogels.

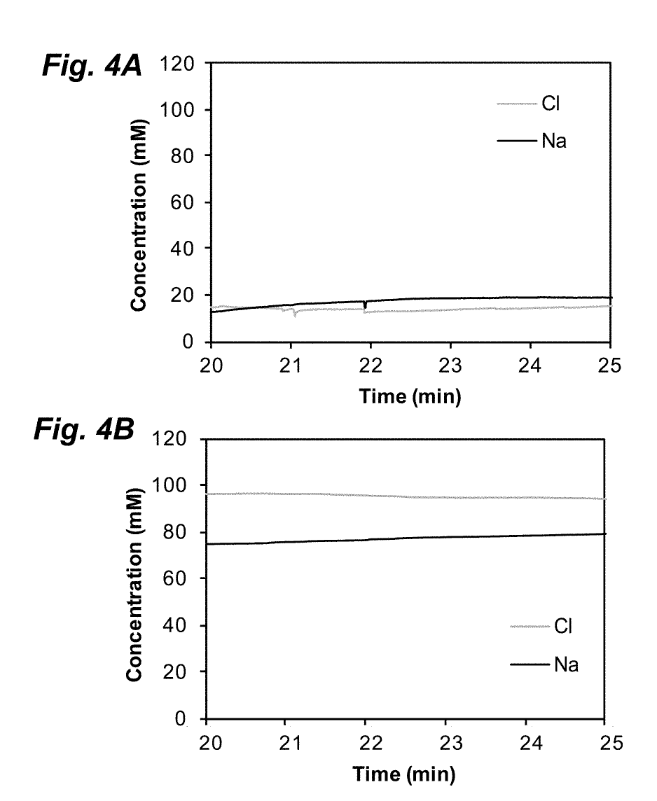

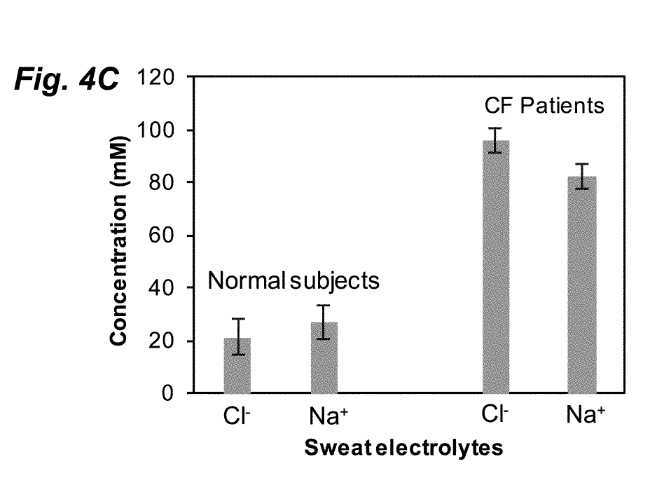

FIG. 4A is a graph of wearable sweat extraction and sensing system for cystic fibrosis diagnosis, showing the real time on body continuous measurement of sweat sodium and chloride levels of a normal subject after iontophoresis based sweat stimulation. FIG. 4B shows the real time measurement of sweat sodium and chloride levels of a CF patient. FIG. 4C shows the comparison of sweat electrolyte levels between a group of normal subjects and a group of CF patients.

This integrated platform can be used both as a diagnostic and clinical investigation tool. To demonstrate its diagnostic capability, the platform was used in the context of cystic fibrosis (CF). As a genetic disease, CF usually leads to an early death and is present in one out of every 3,000 new born Caucasians. Usually sweat test for CF diagnosis is performed by trained technicians, and results are evaluated in an experienced and reliable laboratory over the timespan of hours. For patients that are older than 6 months of age, a chloride level of greater than or equal to 60 mM/L, CF is likely to be diagnosed while the subjects with sweat chloride less than 39 mM/L, CF is very unlikely. It is also known that the normal sweat test and genetic analysis are not always sufficient for some CF patient with rare mutations while the ratio of the sweat sodium and chloride levels can aid the CF diagnosis. Our device can potentially serve as a reliable tool for early diagnosis of cystic fibrosis through on demand sweat stimulation and simultaneous sodium and chloride sensing in sweat. In this case, the wearable system is packaged in a smart wristband and worn by the subjects. A 1 mA current is applied onto the skin for 10 min, which effectively delivers cholinergic agonists to the dermal space to reach the sweat glands and induce sweating. When sweating begins, the sensors measure potential differences between the reference and the working electrodes. The response stabilizes at ˜20 min after iontophoresis, indicating that sufficient sweat has been generated. FIG. 4A and FIG. 4B illustrate the real time on body measurement sweat electrolyte levels for a healthy subject and a CF patient, respectively. It can be clearly observed that both electrolyte levels for the healthy subjects fall below 20 mM while the patient has higher sweat sodium and chloride levels (>60 mM). In situ sweat analysis using our wearable system was performed in six healthy volunteers and three CF patients. As displayed in FIG. 4C, the average sodium and chloride levels for normal subjects are 26.7 and 21.2 mM, respectively, while the average sodium and chloride levels for CF patient subjects are 82.3 and 95.7 mM, respectively. It should be noted that, in agreement with previous report, sweat sodium levels are lower than sweat chloride levels for CF patient subjects in contrast to normal subject where sweat sodium levels are higher, indicating another method to consolidate the diagnostic assessment of CF.

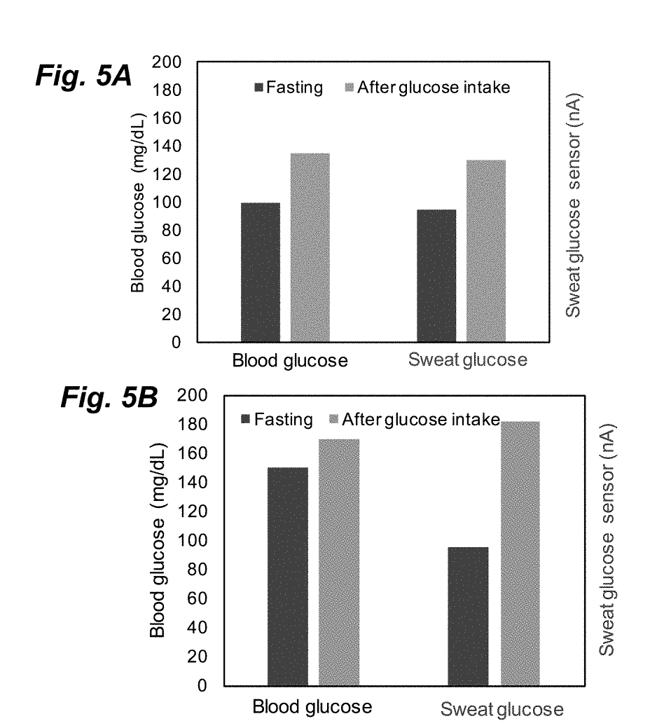

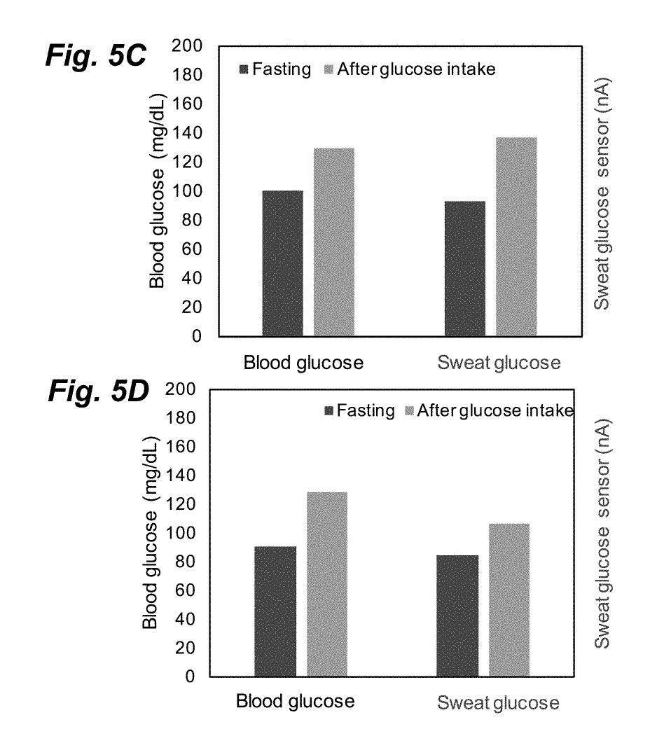

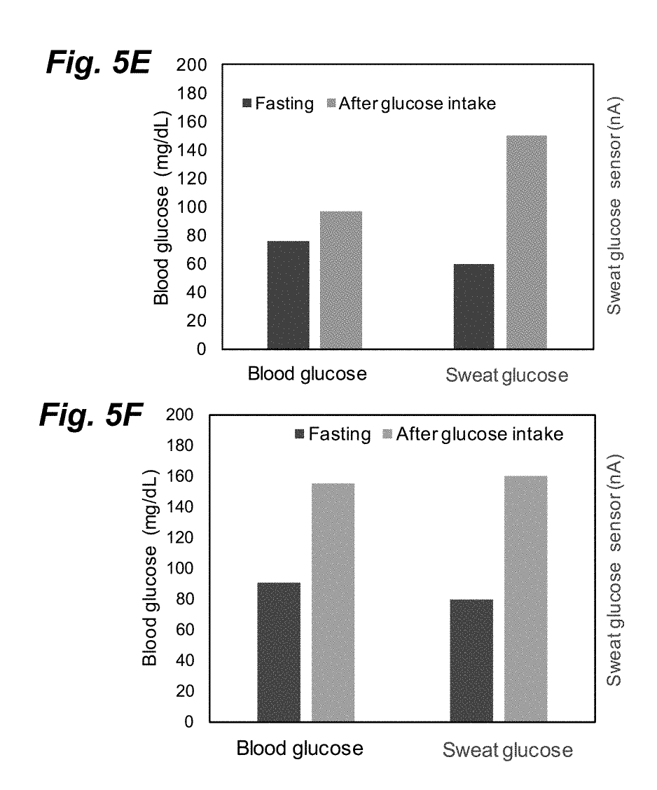

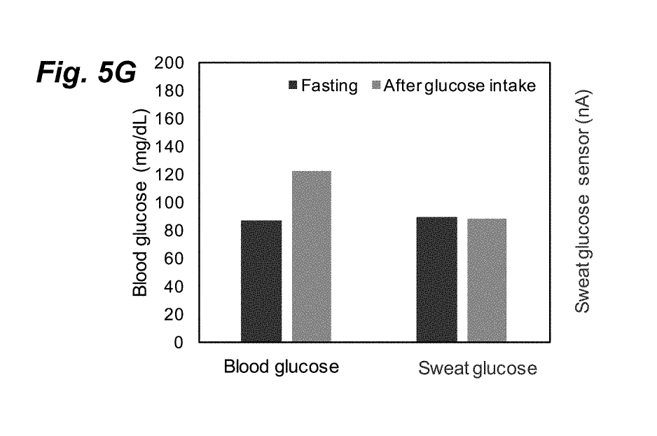

Furthermore, we can use our platform as an investigation tool to enable a wide range of clinical and physiological applications. As an example application, with our platform we conducted preliminary studies toward understanding the metabolic correlation of glucose content in sweat vs. blood. Although there is literature reporting that sweat glucose level is related with blood glucose level, their metabolic correlation has not been well studied. To evaluate the utility of our wearable platform for non-invasive glucose monitoring, real-time sweat stimulation and glucose sensing measurements were conducted on a group of subjects engaged in both fasting and glucose intake trials. FIG. 5A, FIG. 5B, FIG. 5C, FIG. 5D, FIG. 5E, FIG. 5F, FIG. 5G are bar graphs showing the performance of a wearable sweat extraction and sensing system for sweat glucose analysis, specifically comparison of the blood and sweat glucose levels during fasting and 1 hour after glucose intake. The figures illustrate that the sweat and blood glucose levels of seven subjects before and after glucose intake follow similar pattern. Here, the blood glucose analysis is performed using a commercially available glucometer (GE100). The results indicate that oral glucose consumption in fasting subjects results in increase of glucose level in both sweat and blood (from 6 out of 7 subjects). To get more accurate measurements of sweat glucose level and a further understanding on the correlation between sweat and blood glucose levels, embodiments may include the integration of the temperature, pH and sweat rate sensors to calibrate the glucose measurements in sweat.

In this work, we demonstrate a fully integrated and autonomous platform that enables continuous and non-invasive monitoring of individuals through simultaneous extraction (at a high secretion rate) and analysis of sweat, as a physiologically rich yet trivially inaccessible source of information. The device overcomes one of the fundamental challenges of wearable sweat sensing by integrating wirelessly programmable iontophoresis capability to make sweat samples accessible on-demand or at periodic time intervals. Through optimization of sweat stimulating drug concentration in the custom-developed hydrogels and careful design of the iontophoresis electrodes, we were able to consistently achieve secretory rates in excess of 100 nL/cm2/min and extract sufficient amounts of sweat for reliable analysis without causing skin damage or discomfort in the subjects. Additionally, incorporation of simultaneous in-situ analysis functionality inherently allowed for significant reduction of the sweat sample degradation, evaporation or contamination.

To illustrate the value of our solution as a diagnostic tool, we used the platform to detect the elevated sweat sodium and chloride ions content in the Cystic Fibrosis patients. Furthermore, to demonstrate the utility of the platform as a clinical and physiological investigation tool we applied our solution to conduct a preliminary study toward understanding the metabolic correlation of glucose content in sweat vs. blood. Our results indicated that the sweat glucose levels in the fasting subjects increased after oral glucose consumption, in agreement with that observed for the glucose level in blood. To precisely establish the correlation between the sweat and blood glucose, in future, sweat rate monitoring functionality can be integrated to allow for normalization of the analyte content with respect to the sweat rate information of the individual. This added capability is equivalently important for improved quantification and establishment of correlation of other small molecules (e.g. metabolites and proteins), whose abundance in sweat is sweat rate dependent. Furthermore, future efforts will be focused on integration of a wider panel of biomarker, and peripheral electrochemical and physical (e.g. pH and temperature) sensors to deliver a versatile wearable platform for large scale clinical and physiological investigations.

We envision that through enabling such large-scale studies, the device would help to establish the relationship between the sweat profile and the physiological state of the individuals, hence, paving the way for adoption of sweat-based sensing as a non-invasive and seamless method of diagnosis and screening for general population.

Materials and Methods Materials Selectophore grade sodium ionophore X, bis(2-ethylehexyl) sebacate (DOS), sodium tetrakis[3,5-bis(trifluoromethyl)phenyl] borate (Na-TFPB), high-molecular-weight polyvinyl chloride (PVC), tetrahydrofuran, sodium tetraphenylborate (NaTPB), cyclohexanone, polyvinyl butyral resin BUTVAR B-98 (PVB), sodium chloride (NaCl), 3,4-ethylenedioxythiophene (EDOT), poly(sodium 4-styrenesulfonate) (NaPSS), glucose oxidase (from Aspergillus niger), chitosan, single-walled carbon nanotubes, iron (III) chloride, potassium ferricyanide (III), were purchased from Sigma Aldrich (St. Louis, Mo.). Moisture-resistant 100 μm-thick polyethylene terephthalate (PET) was from McMaster-Carr (Los Angeles, Calif.). All reagents were used as received.

### Fabrication of Electrodes Array

The fabrication process includes cleaning polyethylene terephthalate (PET) with IPA and O₂ plasma etching. An electrode array of 3.2 mm in diameter is patterned via photolithography and is thermally evaporated with 30/100 nm of Cr/Au, followed by lift-off in acetone. The electrode array is additionally coated with 500 nm parylene C insulation layer in an SCS Labcoter 2 Parylene Deposition System, and the 3 mm-diameter sensing electrode area is defined via photolithography. The fabricated array is further etched with O₂ plasma to remove the parylene layer at the defined sensing area. Finally, 200 nm Ag is deposited via thermal evaporation and lift-off in acetone. The Ag/AgCl reference electrodes were obtained by injecting 10 μl 0.1-M FeCl₃ solution on top of each Ag reference electrode using a micropipette for 1 min.

Preparation of Na⁺ and Cl⁻ Selective Sensors

The Cl⁻ selective electrode The Na⁺ selective membrane cocktail consisted of Na ionophore X (1% weight by weight, w/w), Na-TFPB (0.55% w/w), PVC (33% w/w), and DOS (65.45% w/w). 100 mg of the membrane cocktail was dissolved in 660 μl of tetrahydrofuran. The ion-selective solutions were sealed and stored at 4° C. The solution for the PVB reference electrode was prepared by dissolving 79.1 mg PVB and 50 mg of NaCl into 1 ml methanol. Poly(3,4-ethylenedioxythiophene) PEDOT:PSS was chosen as the ion-electron transducer to minimize the potential drift of the ISEs and deposited onto the working electrodes by galvanostatic electrochemical polymerization with an external Ag/AgCl reference electrode from a solution containing 0.01M EDOT and 0.1 M NaPSS. A constant current of 14 μA (2 mA cm⁻²) was applied to produce polymerization charges of 10 mC onto each electrode.

Ion-selective membranes were then prepared by drop-casting 10 μl of the Na⁺-selective membrane cocktail onto the corresponding electrodes. The common reference electrode for the Na⁺ and Cl⁻ ISEs was modified by casting 10 μl of reference solution onto the Ag/AgCl electrode. The modified electrodes were left to dry overnight. However, to obtain the best performance, the ion-selective sensors were covered with a solution containing 50 mM NaCl through microinjection for 1 h before measurements. This conditioning process was important to minimize the potential drift.

### Preparation of Glucose Sensors

1% chitosan solution was first prepared by dissolving chitosan in 2% acetic acid and magnetic stirring for about 1 h; next, the chitosan solution was mixed with single-walled carbon nanotubes (2 mg ml⁻¹) by ultrasonic agitation over 30 min to prepare a viscous solution of chitosan and carbon nanotubes. To prepare the glucose sensors, the chitosan/carbon nanotube solution was mixed thoroughly with glucose oxidase solution (10 mg ml⁻¹ in PBS of pH 7.2) in the ratio 2:1 (volume by volume). A Prussian blue mediator layer was deposited onto the Au electrodes by cyclic voltammetry from 0 V to 0.5 V (versus Ag/AgCl) for one cycle at a scan rate of 20 mV s⁻¹ in a fresh solution containing 2.5 mM FeCl₃, 100 mM KCl, 2.5 mM K₃Fe(CN)₆, and 100 mM HCl. The glucose sensor was obtained by drop-casting 3 μl of the glucose oxidase/chitosan/carbon nanotube solution onto the Prussian blue/Au electrode. The sensor arrays were allowed to dry overnight at 4° C. with no light. The solutions were stored at 4° C. when not in use.

### Preparation of Agonist Agent Hydrogels

Hydrogels with cholinergic agonists at different concentrations were prepared based on known methods. In brief, a 3% agarose gel was prepared in a glass beaker by melting the agarose in water for 1 minute in a microwave. The liquefied hot gel was allowed to cool down to 47° C., a magnetic stirrer dropped into the beaker and this was placed on a hot plate stirrer set at 47° C. Then, the appropriate amount of the agonist solution was added to make the desired final concentration and allowed to mix well by stirring for a minute. The melted gel was then poured into a cylindrical mold and allowed to solidify for an hour at 4° C. Next, the hardened gel was sliced in 1 mm disks which were in turn cut to the shape of the iontophoresis electrodes before application to the subject's skin.

### Overall System Design

The overall system was based around the Atmel ATmega328P 8-bit microcontroller with accompanying analog circuitry for both sensor reading and iontophoresis current delivery. The microcontroller's on-board 10-bit analog-to-digital converter (ADC) was used to both read sensor data and to monitor iontophoresis current. A Bluetooth transceiver was connected to the microcontroller to interface the system to a cell phone. Using the cell phone, the system could be commanded to output varying levels of iontophoresis current or to transmit sensor readings in real-time.

### Signal-Conditioning Circuit Design and Processing

Low leakage analog switches were used to interface between the sensors and the beginning of the analog signal-conditioning circuits. The state of these switches was digitally controlled by the microcontroller, and the switches were set to high-impedance (open circuit) during iontophoresis to protect the signal-conditioning circuitry and to minimize the possibility of burning the test subject.

Schematics for the analog signal-conditioning circuitry are shown in FIG. 7A and FIG. 7B, which shows amplifier and low-pass filter circuits. The signal-conditioning circuitry was implemented in relation to the corresponding sensing mode. As shown in FIG. 7A, for the amperometric glucose sensors, the sensor output is in the form of an electrical current, necessitating the use of a transimpedance amplifier (TIA) first stage to amplify the signal and to convert it from a current to a voltage. A 1 MΩ resistor was placed in feedback for the TIA to set the current-to-voltage gain to −106, to allow us to measure current with nanoampere precision. Because the sensor outputs positive current from the Ag/AgCl reference electrode towards the working electrode, and because the TIA has a negative gain, the Ag/AgCl reference electrode was biased to +2.5V to keep the signal within 0-5V range of the microcontroller's ADC. As shown in FIG. 7B, for the potentiometric Na⁺ and Cl⁻ sensors, the sensor output is in the form of a differential voltage. The first stage for the potentiometric sensing channels consisted of Analog Devices AD8422 instrumentation amplifiers with gain set to 5, providing high impedance inputs for the sensors with maximal common-mode noise rejection. By setting the first stage gain to 5, we were able to achieve millivolt-level resolution over the physiologically relevant range of Na⁺ and Cl⁻ concentrations. The PVB reference electrode for the potentiometric sensors was allowed to float, with a 10 kΩ resistor to +2.5 V to provide a path for the input bias current for the amplifiers. The reference terminals of the instrumentation amplifiers were tied to +2.5 V to allow for maximal output swing in single-supply operation.

All of the analog signal-conditioning paths were terminated with a four-pole unity gain low-pass filter, with −3 dB frequency set to 1 Hz to minimize noise and interference in the measurements. The filter outputs were connected to the 10-bit ADC on the microcontroller. ADC readings were oversampled 1000× in software on the microcontroller to further improve resolution and accuracy. These readings were then relayed over Bluetooth to cell phone.

### Iontophoresis Current Delivery and Protection Circuit Design

In order to deliver a wirelessly-controllable iontophoresis current through loads of varying resistance, we designed a current digital to analog converter (DAC) and protection circuitry to interface with the microcontroller. A schematic showing the current delivery circuitry is given in FIG. 8. A second-order low-pass filter followed by voltage buffer was connected to a microcontroller output pin to convert the ATmega328P's pulse-width-modulated (PWM) output to a DC voltage. This voltage was then used to control a voltage-controlled current source, based on an AD8276 difference amplifier with an external bipolar junction transistor (BJT) output stage. This architecture enabled us to use Bluetooth commands to control delivery of iontophoresis currents to the test subject, and allowed us to program iontophoresis currents with arbitrary ramp-up/ramp-down profiles. An ammeter based on the INA282 high-side current shunt monitor was placed in series with the current DAC, and the output was connected to one of the microcontroller's ADC channels to provide real-time monitoring of current delivery, and to enable the microcontroller to shut off current output if excessive current was being drawn. A junction field effect transistor (JFET) and 250Ω series resistor was placed in series with the current path as a safety measure to ensure a maximum short-circuit current of 2 mA. Lastly, analog switches were placed at both positive and negative iontophoresis terminals to fully shut off current when necessary.

### Power Distribution

The system was powered by a single rechargeable lithium-ion polymer battery with a nominal supply voltage of 3.7 V. A single +5 V boost regulator was used to generate the supply voltages for the microcontroller and for the analog signal-conditioning blocks. A +2.5 V virtual ground was used to bias the sensors at mid-supply and to enable efficient, single-supply operation of the analog blocks. A +36 V boost regulator was used to generate the supply voltage for the current DAC, to ensure that the system could deliver appropriate amounts of iontophoresis current through a wide range of physiologically relevant resistive loads. Lastly, a 3.3 V low-dropout (LDO) regulator was used to provide power for the Bluetooth module.

### The Setup of Wearable System for On-Body Testing

A water-absorbent thin rayon pad was placed between the skin and the sensor array during on-body experiments to absorb and maintain sweat for stable and reliable sensor readings, and to prevent direct mechanical contact between the sensors and skin. The on-body measurement results were also consistent with ex situ tests using freshly collected sweat samples.

1. A device for on-demand sweat extraction and analysis, the device comprising:
a printed circuit comprising a microcontroller, an iontophoresis circuit connected to the microcontroller, a sensing circuit connected to the microcontroller, and an electrode array;
wherein the electrode array comprises: iontophoresis electrodes connected to the iontophoresis circuit for sweat induction, and sensing electrodes connected to the sensing circuit for sweat sensing, wherein the sensing electrodes are positioned between the iontophoresis electrodes;
wherein the iontophoresis electrodes comprise a layer of agonist agent hydrogel loaded with sweat stimulating compounds;
wherein the iontophoresis circuit comprises a programmable current source for iontophoresis current delivery,
wherein the sensing circuit includes multiple signal conditioning paths to facilitate multiplexed operation, where each of the signal conditioning paths includes an analog front-end to amplify a sensed signal and a low-pass filter to minimize high frequency noise and electromagnetic interference;
wherein the iontophoresis circuit and the sensing circuit are electrically decoupled for independent functionality.

2. The device of claim 1 wherein the iontophoresis circuit comprises a current protection control circuit.

3. The device of claim 1 wherein the sweat stimulating compounds comprise a cholinergic sweat stimulating compound.

4. The device of claim 1 wherein the iontophoresis electrodes have crescent shapes having convex sides facing each other.

5. The device of claim 1 wherein the iontophoresis electrodes comprise corrosion-proof contacts.
